(19) United States
(12) Patent Application Publication
(10) Pub. No.: US 2021/0345570 A1
(43) Pub. Date: Nov. 11, 2021

(54) VERTICAL-FLOW AGITATION SYSTEM FOR MICROALGAE CULTIVATION TANKS

(71) Applicants: PETRÓLEO BRASILEIRO S.A. - PETROBRAS, Rio de Janeiro (BR); UNIVERSIDADE FEDERAL DE VIÇOSA - UFV, Vicosa (BR)

(72) Inventors: Marcio Aredes Martins, Vicosa (BR); Gerson Ovidio Luz Pedruzi, Vicosa (BR); Emanuele Graciosa Pereira, Vicosa (BR); Adriano Nunes Nesi, Vicosa (BR); Leonardo Brantes Bacellar Mendes, Rio de Janeiro (BR); Rafael Richard Joao, Rio de Janeiro (BR)

(21) Appl. No.: 17/251,679

(22) PCT Filed: Jun. 4, 2019
(86) PCT No.: PCT/BR2019/050204

§ 371 Date: May 19, 2021

(30) Foreign Application Priority Data
Jun. 12, 2018 (BR) BR 102018011846-3

(51) Int. Cl. A01G 33/00; C12M 1/00; C12M 1/02

(52) U.S. Cl. CPC A01G 33/00; C12M 21/02; C12M 27/00; C12M 23/18; C12M 21/12

(57) ABSTRACT

The present invention relates to the cultivation of microalgae for the production of biofuels. In this scenario, the present invention provides a vertical flow agitation system for microalgae culture tanks comprising: a source of energy generation (1); an energy storage device (2); a control system (3); an electric motor (4); at least one end of travel sensor (5); an agitation plate (6); a torque transmission system (7); and at least two lateral drive elements (8).

### FIELD OF THE INVENTION

The present invention relates to the cultivation of microalgae for the production of biofuel. More particularly, the present invention relates to agitation systems of microalgae culture tanks.

### BACKGROUND OF THE INVENTION

The world demand for energy has continued to increase and the obtainment of sources of alternative energy capable of satisfying this increase has become the principal focus of research worldwide. In addition thereto, the intensive utilization of fossil fuels is creating grave problems in relation to environmental degradation, climate change and the health of the population.

Several sources of renewable energy exist which may be exploited to form part of the energy matrix alongside those deriving from petroleum. In this context biofuels have been shown to be a technically and economically viable alternative.

Biodiesel for commercial use has been produced from vegetable oils, from waste oils from frying and from animal fats. Vegetable oils are important sources of triacylglycerides employed in the production of biodiesel both by virtue of the quality of the profile of fatty acids and by virtue of the availability of these oils in the agricultural sector.

However, the production of biodiesel from vegetable oils, such as of soya, sunflower and cotton, competes with the production of foods by virtue of the fact that the principal vegetable crops producing these oils also supply other products for the food industry. Consequently, it is necessary to identify and develop a source of triglycerides not of interest to other industrial sectors, with the objective of minimizing competition against these sectors, thus ensuring the viability of biodiesel as an alternative source to diesel. In this context, microalgae are presented as a very promising source of oil for the production of biodiesel.

In the same manner as vascular plants, microalgae require three basic components for growth: light, water and nutrients. In addition, microalgae possess a photosynthetic efficiency exceeding that of vascular plants, with rapid growth and accumulation of vegetable biomass, that is to say that they produce more biomass per hectare in less time.

The expected oil/hectare productivity of microalgae exceeds by approximately 10 times the productivity of palm, for example, this being considered to be the most productive land species in terms of oil. Consequently, microalgae are an alternative with significant potential for the production of biofuels (biomass, oil, biodiesel, methane and hydrogen).

In addition, by virtue of the rapid growth thereof, microalgae are efficient fixers of atmospheric carbon, fixing large volumes of carbon, by means of photosynthesis, in a very short time. It is estimated that each tonne of algal biomass produced consumes approximately two tonnes of carbon dioxide through photosynthesis, this representing from ten to twenty times more carbon than that absorbed by oleaginous crops.

A further advantage observed resides in the fact that the production of biodiesel from microalgae does not compete with the food industry, by virtue of the fact that it requires less extensive areas for cultivation and it may be carried out in areas which are not of interest for agriculture.

By virtue of the fact that they naturally act as fixers of carbon dioxide, the cultivation of microalgae may be associated with lines of emission of carbon dioxide from industry, such as cement factories, oil refineries, paper, cellulose, and iron and steel plants and thermal power units, these being the large emitters of carbon dioxide.

The fixation of carbon dioxide of itself would add value to these industries through the carbon credit market. In addition to the fixation of carbon, microalgae possess a great capacity for the removal of nutrients from waste water, such as petrochemical waste water, assisting in the treatment of effluents.

The commercial production of microalgae is carried out both in open and in closed systems. Closed systems, denominated photobioreactors, are those wherein direct contact does not exist between the culture and the external environment. In these systems the risk of contamination is lower and there is greater control over process conditions, such as temperature, pH and concentration of nutrients.

On the other hand, photobioreactors are characterized by high ratios of surface area to volume and by requiring cooling devices having high energy consumption, this hindering the application thereof in production on a large scale. In the system of cultivation in open ponds there is direct contact with the external environment, rendering it more vulnerable to contamination.

Furthermore, the ratio between surface area and volume presents moderate values and the costs of operation and maintenance of this type of system are much lower than the values found in closed systems, rendering them more attractive for large scale production. But even in the system of production of microalgae in open ponds there exists the need to stir the culture to expose the cells to the light, demanding energy.

Currently, the type of open technique utilized for large scale production of microalgae is denominated a raceway system, generally constituted by a masonry structure having a form being elliptical, shallow and divided in the middle such as to form two parallel channels, one thereof provided with an agitator for moving the biomass in suspension. The agitator is generally constituted by means of submerged pumping, air injection or by paddle wheels.

The raceway system presents a high demand for electrical energy for the operation thereof because the agitators thereof maintain the biomass in suspension in order to expose the cells thereof to the light and, furthermore, they promote a circular movement of the entirety of the fluid located in the pond. In this manner there is a high demand for energy in order to bring about the efficient agitation of this type of system and, consequently, additional costs are incurred for the final product.

The vertical flow agitation system is another technique utilized to expose the cells of the culture. The agitator presents the form of a vertical structure which, disposed within the tank, divides the total volume of the same into two contiguous sections of variable volume, interconnected solely by a narrow communication at the inferior extremity thereof. The vertical structure traverses the entire tank in the longitudinal direction thereof and repeats this process in continuous cycles. This system demands less energy than does the raceway system, reducing the cost of the production of the algal biomass, nevertheless in a large scale cultivation system the use of electrical energy is still required.

Diverse systems of agitation applied to tanks of microalgae cultivation are known in the state of the art. Some of these systems are presented below.

The document US7763457B2 describes a system of cultivation of algae for use as a source of biodiesel, comprising barriers separated from one another by predetermined distances such as to create a wake of von Kármán vortices in order to move the algae cells, sequentially, to the surface of the flow to receive solar light. In order to bring about the flow through the channels paddle wheels are utilized, making the fluid move through the channels.

The document WO2013153402A1 reveals a method of cultivating microalgae in raceway type ponds, wherein the physiological state of the algae is manipulated through the alteration of one or more environmental parameter such as to simulate conditions of blooming of the algae and the conditions of the pond itself. The alteration of one or more environmental parameters in a specifically timed manner may be employed to induce and maintain synchronous cellular division. According to this document, in order to maintain the flow of microalgae and water circulating through the pond paddle wheels are adopted, located at the extremities of the ponds.

The document WO2008048861A2 proposes a system of production of algae for use in a two stage reactor, comprising an algae separator connected by a conduit to a cultivation reactor destined for the growth of algae having a high oil content.

According to WO2008048861A2, in order that the flow of the microalgae culture be maintained constant in a plug flow reactor paddle wheels are utilized in a second reactor. However, no details are furnished in relation to these devices.

The document U.S. Pat. No. 9,593,302B1 reveals a method for the fractionation of a microalgae culture, comprising additions of culture medium in aqueous phase to a tank, transferring the growing culture to a device having the objective of removing the upper fraction and collecting the bottom fraction containing microalgae. The tank described comprises a device to move the culture, wherein this device may be, inter glia, a mixer, a pump, a set of paddles, without any details being furnished.

The document CN203668406U reveals a device to control agitation by means of the injection of air into a culture of microalgae in a tank, comprising a gas distributor configured with an aeration manifold provided with an electromagnetic valve. According to this document, the agitation control assembly comprises a solar panel, a first resistance, a frequency converter, a motor, and a rotary agitation paddle.

In the light of that stated it is clear that the state of the art still presents a demand for automated and autonomously controlled systems for vertical flow agitation systems applied to microalgae culture tanks, or to any reactor making use of this type of agitation system, with the objective of reducing the cost of production of microalgae biomass for the purpose of the production of biodiesel.

The present invention seeks to solve the aforedescribed problems in the state of the art in a practical and efficient manner, being described in detail in the following section.

### SUMMARY OF THE INVENTION

The objective of the present invention is to provide vertical flow agitation systems applied to tanks of microalgae cultivation, or to any bioreactor, being more efficient than those known in the state of the art, in addition to providing a reduction in the consumption of energy of these systems.

In order to achieve the aforedescribed objectives, the present invention provides a vertical flow agitation system for microalgae culture tanks comprising: a source of energy generation; an energy storage device; a control system; an electric motor; at least one end of travel sensor; an agitation plate; a torque transmission system; and at least two lateral drive elements.

### BRIEF DESCRIPTION OF THE DRAWINGS

The detailed description presented in the following section makes reference to the appended FIGURES and the respective reference numbers thereof.

FIG. 1 illustrates a view of an optional configuration of the autonomous vertical flow agitation system of the present invention.

### DETAILED DESCRIPTION OF THE INVENTION

In a preliminary manner it is emphasized that the description which follows arises from a preferential embodiment of the invention. As will however be obvious to anyone skilled in the art the invention is not limited to this particular embodiment.

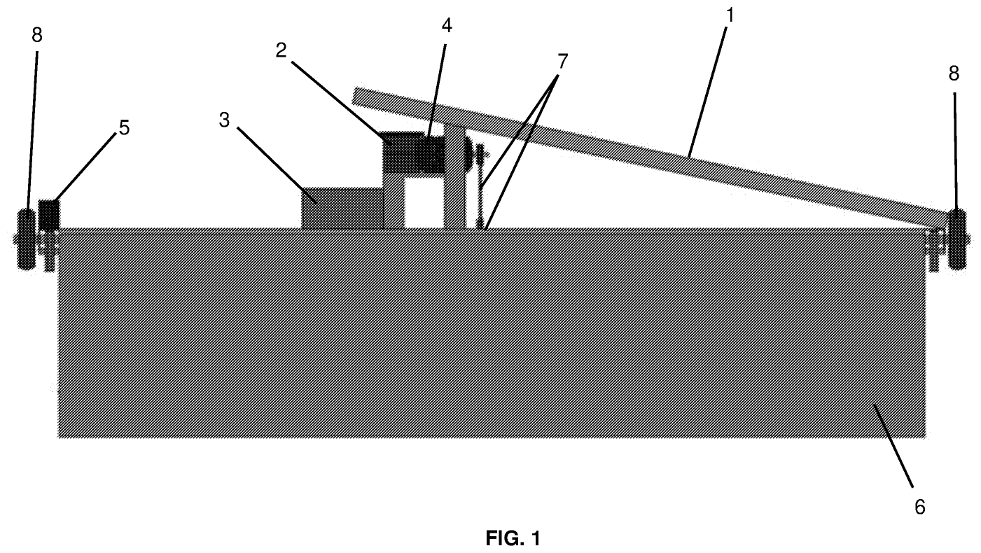

FIG. 1 illustrates a view of a particular configuration of the autonomous vertical flow agitation system of the present invention.

As may be observed in FIG. 1, in a specific configuration of the invention herein described, the system comprises: a source of energy generation 1; an energy storage device 2; a control system 3; an electric motor 4; at least one end of travel sensor 5; an agitation plate 6; a torque transmission system 7; and at least two lateral drive elements 8.

The source of energy generation 1 is preferentially an autonomous generation source providing all the energy required for the operation of the system. Diverse sources of energy generation 1 may be adopted, wherein an autonomous source is preferred.

Optionally, the source of energy generation 1 is of the renewable type and may be of any type known in the state of the art such as, inter glia, photovoltaic or wind energy generators.

In FIG. 1 a configuration is illustrated wherein the source of energy generation 1 is a photovoltaic plate positioned above the autonomous vertical flow agitation system. In this configuration solar energy could be transformed into electrical energy to supply the entire system.

The invention furthermore anticipates a combination of different sources of energy, consequently, should the environmental conditions not be favorable for one type of source of energy generation 1 others may be used, not compromising the overall operation of the system.

The energy storage device 2 is optionally adopted to store the excess energy generated by the source of energy generation 1. The energy storage device 2 adopted may be any known in the state of the art, such as at least one battery, at least one supercapacitor, or the interaction of these latter, among others.

Optionally, as illustrated in FIG. 1, the energy storage device 2 is incorporated into the assembly of the vertical flow agitation system. In alternative configurations, however, the energy storage device 2 may be fixed at a point external to the system and connected to the same by electrical wiring.

Optionally, there is also adopted a control system 3 executing the control over the direction of movement of the autonomous vertical flow agitation system. This control system 3 may be composed by microcontrollers or by programmable logic controllers, or by relays, or by the interaction of these latter, among others.

Furthermore, when adopted, the control system 3 may be affixed to the assembly of the vertical flow agitation system. Nevertheless, in alternative configurations, the control system 3 may be fixed at a point external to the system, wherein the communication between the control system 3 and the elements controlled may be realized in wireless form.

The end of travel sensors 5 are adopted to detect when the agitation system reaches the end of the culture tank in one direction. When this occurs, the end of travel sensor 5 sends data to the control system 3 which will reverse the movement of the agitation system, it being displaced in the opposite direction, that is to say towards the other extremity of the culture tank.

It is emphasized that the control system 3 is responsible for controlling all the elements of the agitation system, wherein the communication between the control system and the other elements may be realized in any known manner, such as by electrical wiring or wireless connections.

The motor 4 is the device which will transform the electrical power received from the generating source 1, or from the storage device 2, into mechanical work to actually drive the agitation system, in particular, the agitation plate.

For this purpose, the invention comprises a transmission system 7 to transmit the torque generated by the motor 4 to the drive elements 8.

The transmission system 7 adopted may be any known in the state of the art. In the optional configuration illustrated, the transmission system 7 comprises a set of gears and shafts transmitting the required torque to the drive elements 8.

The drive elements 8 responsible for moving the agitation system, in particular the agitation plate 6, along the culture tank. Optionally, the drive elements 8 are positioned laterally in relation to the agitation plate 6, in this manner they also assist in the physical equilibrium of the system.

It is emphasized that, in spite of the fact that the lateral drive elements 8 illustrated in FIG. 1 comprise two wheels, each one positioned at each lateral extremity of the agitation plate 6, other configurations may be adopted.

For example, the drive elements 8 may comprise lateral rails or lateral belts connected to toothed shafts, or any other configurations permitting the driving of the agitation system along the culture tank.

Optionally, the drive elements 8 may be supported and be moved upon a lateral rim of the culture tank.

The agitation plate 6 is the element responsible for actually realizing the agitation of the liquid (and, consequently, of the microalgae) in the culture tank, such that the microalgae cells may be always exposed to the light.

The agitation plate 6 adopted is of the type commonly adopted in the state of the art. Consequently, the agitation plate 6 has lateral dimensions approximately equal to the internal lateral dimensions of the culture tank such that the minimum flow of culture is permitted to pass the sides of the agitation plate 6.

In addition, the agitation plate 6 extends vertically from a position above the water line of the culture tank to a region proximate to the bottom of the tank.

In this manner, the agitation plate 6 may present different formats, wherein the format adopted must substantially respect the format of the cross-section of the culture tank, wherein the sides of the agitation plate 6 must be located as proximately as possible to the walls of the tank, and the inferior region of the agitation plate 6 must maintain a given distance from the bottom of the tank such as to permit the passage of fluid past the inferior region.

Consequently, the operation of the vertical flow agitation system of the present invention may be summarized as described below.

The source of energy generation 1 furnishes the energy for the entire system and directs at least part of the energy to the storage device 2, to the motor 4 and to the control system 3. The motor 4 furnishes the mechanical torque to the transmission system 7 which transmits torque to the drive elements 8 and moves the system.

The control system 3 defines the direction of movement of the whole assembly shown in FIG. 1, that is to say when the same reaches one of the extremities of the tank the end of travel sensor 5 detects the position thereof and sends a signal to the control system 3 in order for the same to reverse the rotation of the motor 4. The assembly is then displaced in the contrary direction, in this manner executing a periodic movement, travelling along the entire tank in the longitudinal direction.

In this manner, whilst the system is being displaced in the culture, the agitation plate 6, being in direct contact with the same, executes the agitation of the microalgae, exposing the same to the light.

Consequently, the invention provides an autonomous vertical flow agitation system through the utilization of a photovoltaic generation unit to furnish all the energy required to execute the agitation of the culture, generating a reduction in the cost of electrical energy.

By means of all that set out it is clear that the vertical flow agitation system of the present invention demonstrates a series of advantages in relation to the models of the state of the art, they being: reduction in the unit cost of production of biomass from microalgae; automation of the agitation system, rendering it capable of operation even in remote areas whereat the offer of electrical energy is non-existent; low maintenance system, by virtue of the fact that the devices comprising it possess high durability; and facility of adaptation of the current culture systems to this invention.

Innumerable variations affecting the scope of protection of the present application are permissible. In this manner, the fact is reinforced that the present invention is not limited to the aforedescribed specific configurations and embodiments.

1. A vertical flow agitation system for microalgae culture tanks, wherein it comprises: a source of energy generation (1); an energy storage device (2); a control system (3); an electric motor (4); at least one end of travel sensor (5); an agitation plate (6); a torque transmission system (7); and at least two lateral drive elements (8).

2. A system as claimed in claim 1, wherein the source of energy generation (1) is at least one of: a photovoltaic energy generator, and a wind energy generator.

3. A system as claimed in claim 2, wherein the energy storage device (2) is at least one battery, or at least one supercapacitor, or a combination thereof.

4. A system as claimed in any one of the claims 1 to 3, wherein the control system (3) is adapted to execute the control of the direction of movement of the autonomous vertical flow agitation system, wherein the control system (3) is composed by at least one of: microcontrollers, programmable logic controllers, and relays.

5. A system as claimed in any one of the claims 1 to 4, wherein the end of travel sensors (5) are adapted to detect when the agitation system reaches the end of the culture tank in a given direction.

6. A system as claimed in any one of the claims 1 to 3, wherein the torque transmission system (7) is adapted to transmit the torque generated by the motor (4) to the drive elements (8), wherein the transmission system (7) comprises a set of gears and shafts.

7. A system as claimed in any one of the claims 1 to 6, wherein
the drive elements (8) are adapted to drive the agitation system along the culture tank,
wherein the drive elements (8) are positioned laterally in relation to the agitation plate (6),
wherein the drive elements (8) comprise at least one of: lateral rails, lateral belts connected to toothed shafts, and lateral wheels, and
wherein the drive elements (8) are supported and move upon a lateral rim of the culture tank.

8. A system as claimed in any one of the claims 1 to 7, wherein the agitation plate (6) comprises a format substantially respecting the format of the cross-section of the culture tank, wherein the sides of the agitation plate (6) are positioned in a precise manner in relation to the lateral walls of the tank, and the inferior region of the agitation plate (6) maintains a given distance from the bottom of the tank such that the passage of fluid is permitted past the inferior region.
